(19) United States
(12) Patent Application Publication
(10) Pub. No.: US 2025/0340815 A1
(43) Pub. Date: Nov. 6, 2025

(54) MODULAR SPARGER ASSEMBLY FOR A BIOPROCESSING SYSTEM

(71) Applicant: Global Life Sciences Solutions USA LLC, Mariborough, MA (US)

(72) Inventors: Kevin Ashley Lamport, Portsmouth Hampshire (GB); Matthew Ouellette, Marlborough, MA (US); Timothy Becker, Marlborough, MA (US); Paul Seeto, Marlborough, MA (US); Michael Jason Miller, Marlborough, MA (US); Sree Bandaru, Marlborough, MA (US); Ivan Reed, Portsmouth Hampshire (GB)

(73) Assignee: Global Life Sciences Solutions USA LLC, Marlborough, MA (US)

(21) Appl. No.: 18/654,069

(22) Filed: May 3, 2024

(51) Int. Cl. C12M 1/00

(52) U.S. Cl. CPC C12M 29/06

(57) ABSTRACT

A sparging assembly includes a first base plate having at least one mounting element for receiving a first sparger device, and a second base plate having at least one mounting element for receiving a second sparger device. The first base plate and the second base plate are mechanically separate from one another.

### BACKGROUND

### Technical Field

Embodiments of the invention relate generally to bioprocessing systems and methods and, more particularly, to a modular sparger assembly for a bioprocessing system.

### Discussion of Art

A variety of vessels, devices, components and unit operations are known for carrying out biochemical and/or biological processes and/or manipulating liquids and other products of such processes. In order to avoid the time, expense, and difficulties associated with sterilizing the vessels used in biopharmaceutical manufacturing processes, single-use or disposable bioreactor bags and single-use mixer bags are used as such vessels. For instance, biological materials (e.g., animal and plant cells) including, for example, mammalian, plant or insect cells and microbial cultures can be processed using disposable or single-use mixers and bioreactors.

In the biopharmaceutical industry, single use or disposable containers are often used for bioprocessing operations. Such containers can be flexible or collapsible plastic bags that are supported by an outer rigid structure such as a stainless steel shell or vessel. Use of sterilized disposable bags eliminates time-consuming step of cleaning of the vessel and reduces the chance of contamination. The bag may be positioned within the rigid vessel and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the bag/vessel.

Depending on the fluid being processed, the bioreactor system may include a number of fluid lines and different sensors, probes and ports coupled with the bag for monitoring, analytics, sampling, and liquid transfer. For example, a harvest port is typically located at the bottom of the disposable bag and the vessel, and allows for a harvest line to be connected to the bag for harvesting and draining of the bag. In addition, existing bioreactor systems typically utilize spargers for introducing a controlled amount of a specific gas or combination of gases into the bag. A sparger outputs small gas bubbles into a liquid in order to agitate and/or dissolve the gas into the liquid, or for carbon dioxide stripping. The delivery of gas via spargers helps in mixing a substance, maintaining a homogenous environment throughout the interior of the bag, and is sometimes essential for growing cells in a bioreactor.

Ideally, the spargers and the agitator/impeller are in close proximity to ensure optimal distribution of the gases throughout the liquid volume. Most typically, spargers are integrated directly into the agitator/impeller base plate in the case of bottom-driven agitators and, at most, have a small number of fixed locations on the impeller base plate where the sparger elements can be mounted. This arrangement, however, does not allow for much, if any, flexibility in the positioning or layout of the sparger elements within the flexible bag.

In addition, existing sparger configurations and mounting arrangements can be prone to cyclic fatigue, compromising sparger integrity. In particular, fluid moved by the impeller during extended operation can exert a drag force on the tubing that supplies the sparger elements with gas, causing oscillations which then propagate to the sparger elements and the mounting fixtures which connect them to the impeller base plate.

In view of the above, there is a need for a modular sparger assembly and tubing fixturing arrangement that allows for the customization of sparger layout in view of customer and/or application demands, and which minimizes the possibility of cyclic fatigue loading of sparger mounting fixtures.

### Brief Description

In an embodiment, a sparging assembly is provided. The sparging assembly includes a first base plate having at least one mounting element for receiving a first sparger device, and a second base plate having at least one mounting element for receiving a second sparger device. The first base plate and the second base plate are mechanically separate from one another.

In another embodiment, a bioprocessing system is provided. The bioprocessing system includes a bioprocessing container; a plurality of base plates mounted within an interior of the bioprocessing container, at least one of the plurality of base plates being mechanically separate from another of the plurality of base plates, and a plurality of sparger devices mounted to the plurality of base plates.

In yet another embodiment, a method of configuring a bioprocessing system is provided. The method includes the steps of affixing an impeller base plate to an interior of a flexible bioprocessing bag, mounting an impeller to the impeller base plate, affixing a first sparger base plate to the interior of the flexible bioprocessing bag adjacent to the impeller base plate, and mounting a first sparger device to the first sparger base plate. The first sparger base plate and the impeller base plate are spaced from one another.

### BRIEF DESCRIPTION OF DRAWINGS

The present invention will be better understood from reading the following description of non-limiting embodiments, with reference to the attached drawings, wherein below:

FIG. 1 is a front elevational view of a bioprocessing system according to an embodiment of the invention.

FIG. 2 is a simplified side elevational, cross-sectional view of the bioprocessing system of FIG. 1.

FIG. 3 is a perspective view of a prior art impeller base plate.

FIG. 4 is a perspective view of an impeller base plate according to an embodiment of the invention.

FIG. 5 is a sparger base plate according to an embodiment of the invention.

FIG. 6 is a perspective view of a sparger element according to an embodiment of the invention.

FIG. 7 is a perspective view of a modular sparger assembly according to an embodiment of the invention.

FIG. 8 is a perspective view of a modular sparger assembly according to another embodiment of the invention.

FIG. 9 is a perspective view of a modular sparger assembly according to an embodiment of the invention.

FIG. 10 is a perspective view of a modular sparger assembly according to another embodiment of the invention.

FIG. 11 is a perspective view of a modular sparger assembly according to an embodiment of the invention.

FIG. 12 is a perspective view of a module sparger assembly and tubing fixturing arrangement according to an embodiment of the invention.

FIG. 13 is a perspective view of a module sparger assembly and tubing fixturing arrangement according to another embodiment of the invention.

FIG. 14 is an enlarged, perspective view of a tubing fixturing system according to an embodiment of the invention.

FIG. 15 is an enlarged, perspective view of a portion of the tubing fixturing system of FIG. 14.

FIG. 16 is an enlarged, perspective view of another portion of the tubing fixturing system of FIG. 14.

FIG. 17 is an enlarged, perspective view of a tubing fixturing system according to an embodiment of the invention.

FIG. 18 is an enlarged, perspective view of a tubing fixturing system according to another embodiment of the invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, a rigid container, or a flexible or semi-rigid tubing, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems. As used herein, the term “bag” means a flexible or semi-rigid container or vessel used, for example, as a bioreactor or mixer for the contents within.

Embodiments of the invention provide sparging assemblies for a bioprocessing system. In an embodiment, a sparging assembly includes a first base plate having at least one mounting element for receiving a first sparger device, and a second base plate having at least one mounting element for receiving a second sparger device, wherein the first base plate and the second base plate are mechanically separate from one another. The sparging assembly may also include an impeller base plate having a mounting mechanism for receiving an impeller or agitator, whereby the impeller base plate is mechanically separate from at least one of the first base plate and the second base plate.

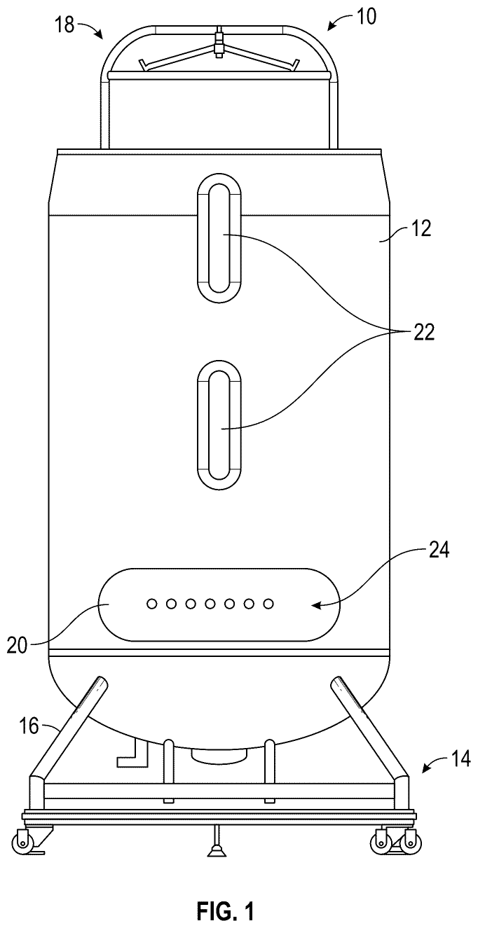

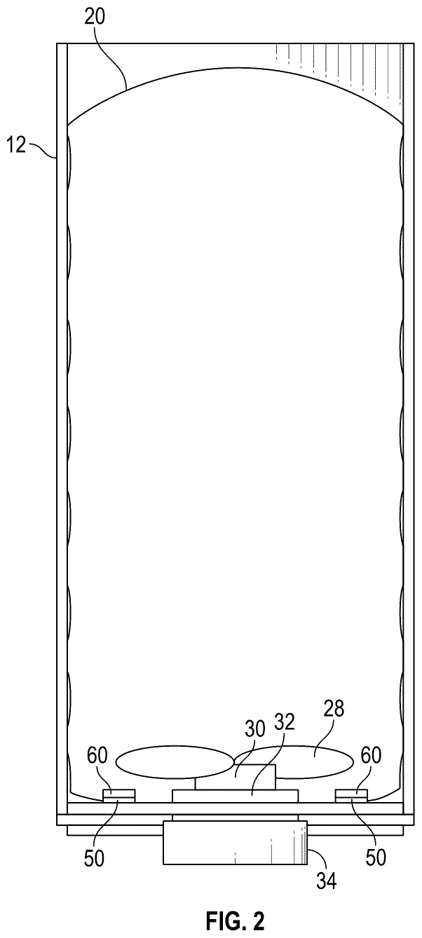

With reference to FIGS. 1 and 2, a bioprocessing system 10 according to an embodiment of the invention is illustrated. The bioprocessing system 10 includes a generally rigid bioreactor vessel or support structure 12 mounted atop a base 14 having a plurality of legs 16. The vessel 12 may be formed, for example, from stainless steel, polymers, composites, glass, or other metals, and may be cylindrical in shape, although other shapes may also be utilized without departing from the broader aspects of the invention. The vessel 12 may be outfitted with a lift assembly 18 that provides support to a single-use, flexible bag 20 disposed within the vessel 12. The vessel 12 can be any shape or size as long as it is capable of supporting a single-use flexible bioreactor bag 20. For example, according to one embodiment of the invention the vessel 12 is capable of accepting and supporting a 10-2000L flexible or collapsible bioprocess bag assembly 20.

The vessel 12 may include one or more sight windows 22, which allows one to view a fluid level within the flexible bag 20, as well as a window 24 positioned at a lower area of the vessel 12. The window 24 allows access to the interior of the vessel 12 for insertion and positioning of various sensors and probes (not shown) within the flexible bag 20, and for connecting one or more fluid lines to the flexible bag 20 for fluids, gases, and the like, to be added or withdrawn from the flexible bag 20. Sensors/probes and controls for monitoring and controlling important process parameters include any one or more, and combinations of: temperature, pressure, pH, dissolved oxygen (DO), dissolved carbon dioxide (pCO₂), mixing rate, and gas flow rate, for example.

With specific reference to FIG. 2, a schematic side elevational, cutaway view of the bioprocessing system 10 is illustrated. As shown therein, the single-use, flexible bag 20 is disposed within the vessel 12 and restrained thereby. In embodiments, the single-use, flexible bag 20 is formed of a suitable flexible material, such as a homopolymer or a copolymer. The flexible material can be one that is USP Class VI certified, for example, silicone, polycarbonate, polyethylene, and polypropylene. Non-limiting examples of flexible materials include polymers such as polyethylene (for example, linear low density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. In an embodiment, the flexible material may be a laminate of several different materials such as, for example Fortem™, Bioclear™10 and Bioclear 11 laminates, available from Cytiva. Portions of the flexible container can comprise a substantially rigid material such as a rigid polymer, for example, high density polyethylene, metal, or glass. The flexible bag may be supplied pre-sterilized, such as using gamma irradiation.

The flexible bag 20 contains an impeller 28 attached to a magnetic hub 30 at the bottom, center of the inside of the bag, which rotates on an impeller base plate 32 also positioned on the inside bottom of the bag 20. Together, the impeller 28 and hub 30 (and in some embodiments, the impeller plate) form an impeller assembly. A magnetic drive 34 external to the vessel 12 provides the motive force for rotating the magnetic hub 30 and impeller 28 to mix the contents of the flexible bag 20. While FIG. 2 illustrates the use of a magnetically-driven impeller, other types of impellers and drive systems are also possible, including top-driven impellers.

As also illustrated in FIG. 2, the bottom of the flexible bag 20 includes a plurality of sparger base plates 50 affixed thereto, to which one or more sparge pods 60 (also referred to herein sparger devices or sparger elements) are mounted. The sparge pods 60 are configured for connection to a supply of gas via a port in the bottom or sidewall of the flexible bag 20 and tubing extending form the port to the sparge pods 60, as described in detail hereinafter. The sparge pods 60 extend upwardly into the interior volume defined by the flexible bag 20 and are positioned adjacent to the impeller 28.

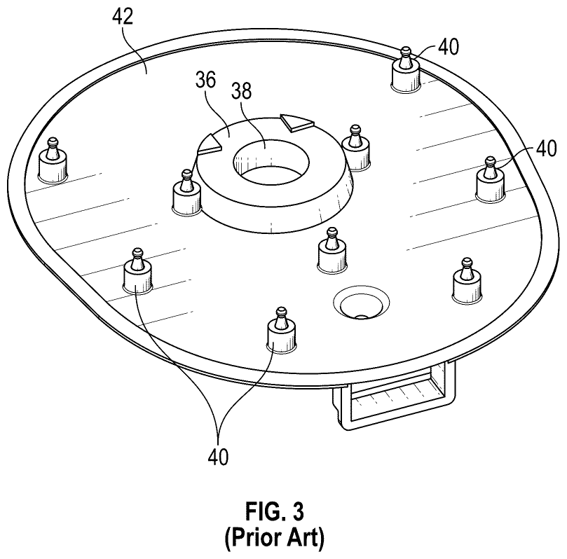

As described in detail hereinafter, the sparger base plates 50 and sparge pods 60 are mechanically separate from, and spaced around, the impeller base plate 32 and impeller 28, which allows the sparge pods 60 to be arranged in an almost infinite number of positions at the bottom of the flexible bag 20. This is in contrast to existing systems, where sparge pods are typically integrated directly into the impeller base, which has a small number of fixed locations where the sparger elements can be mounted. FIG. 3 shows an example of a prior art impeller base plate 42 having fixed locations for sparge pod mounting. As shown therein, the impeller base plate 42 has a central ring 36 having a central opening 38 atop which the impeller 28 is positioned for rotation. The base plate 42 also includes a plurality of mounting posts 40 arranged about the central ring 36 which provide fixed locations around the central ring 36 where sparge pods can be mounted. Existing impeller base plates 42 thus serve as the base or support structure to which both the impeller 28 and the sparge pods are mounted.

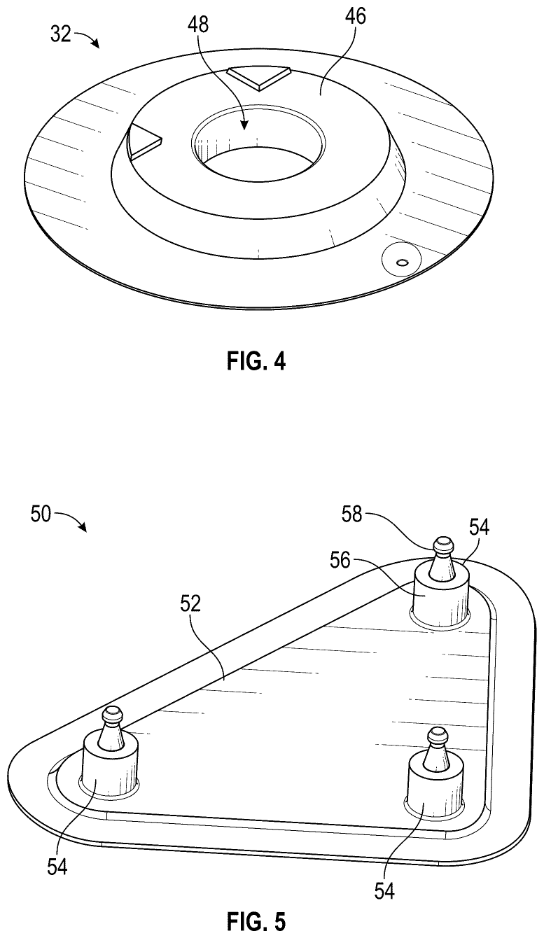

As indicated above, however, embodiments of the invention provide separate impeller and sparge pod base plates, which allows for greater flexibility in the location/positioning of the sparge pods 60 within the processing volume of the bioprocessing system 10. FIG. 4 illustrates an exemplary impeller base plate 32. As shown therein, the base plate 32 has a central hub 46 extending upwardly from the base plate, having a central opening 48 atop which the impeller 28 is positioned for rotation. The impeller base plate 32 is affixed to the interior bottom of the flexible bag 20 in a manner heretofore known in the art, and is typically located centrally within the bag 20, although other mounting locations are also possible. While not shown in FIG. 4, the impeller base plate 32 may include a plurality of mounting posts for mounting one or more sparge pods 60 thereto, as disclosed hereinafter. Moreover, while FIG. 4 shows the impeller base plate 32 as being circular in shape, the invention is not intended to be so limited in this regard, and the impeller base plate 32 may be configured in a variety of shapes such as elliptical, oblong, triangular, rectangular, irregularly shaped, and the like, without departing from the broader aspects of the invention.

Turning now to FIG. 5, a sparger base plate 50 according to an embodiment of the invention is illustrated. As shown therein, the sparger base plate 50 includes a generally flat or planar body 52 and a plurality of mounting posts 54 extending upwardly therefrom. In an embodiment, there are three mounting posts 54 arranged in a triangular configuration, however, the arrangement and number of mounting posts may be varied depending on the particular configuration of the sparge pods. As further shown therein, in an embodiment, the mounting posts 54 have a wide, generally cylindrical base 56, and a narrower distal end 58. While FIG. 5 illustrates the sparger base plates 50 as being triangular in shape, the invention is not intended to be so limited in this regard, and it is contemplated that the base plates 50 can be configured in a variety of different shapes.

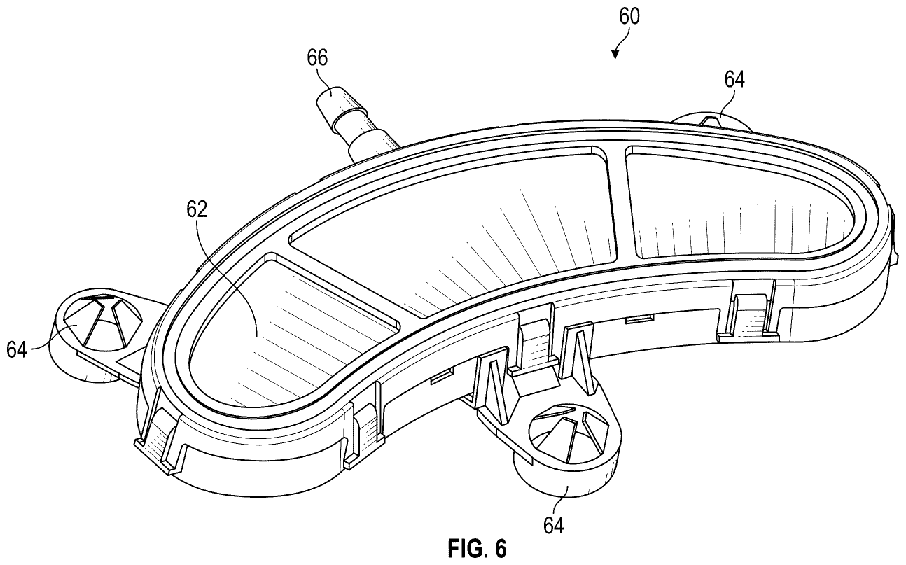

With reference to FIG. 6, sparge pods 60 may take the form of any sparger known in the art, and include a body portion 62 and a plurality of mounting members in the form of feet 64. The feet 64 are arranged in a configuration that corresponds to the configuration of the mounting posts 54 on the sparger base plate 50 (e.g., a triangular configuration), and are configured to be received on the sparger base plate 50 via a snap fit connection. As also shown in FIG. 6, the sparge pods 60 also include a tubing connector 66 for the connection of sparge tubing thereto, which provides the sparge pods 60 with a supply of sparge gas that is distributed to the processing volume within the flexible bag 20 by the sparge pod 60. As indicated above, the sparge pods 60 may be any type of sparger known in the art and are configured to create small bubbles and/or disperse gas evenly throughout the flexible bag 20 to promote efficient mixing, aeration and/or chemical reactions. For example, in an embodiment, the body portion of the sparge pods 60 is a sintered porous material made of metal or ceramic. Other types of spargers known in the art may also be utilized without departing from the broader aspects of the invention.

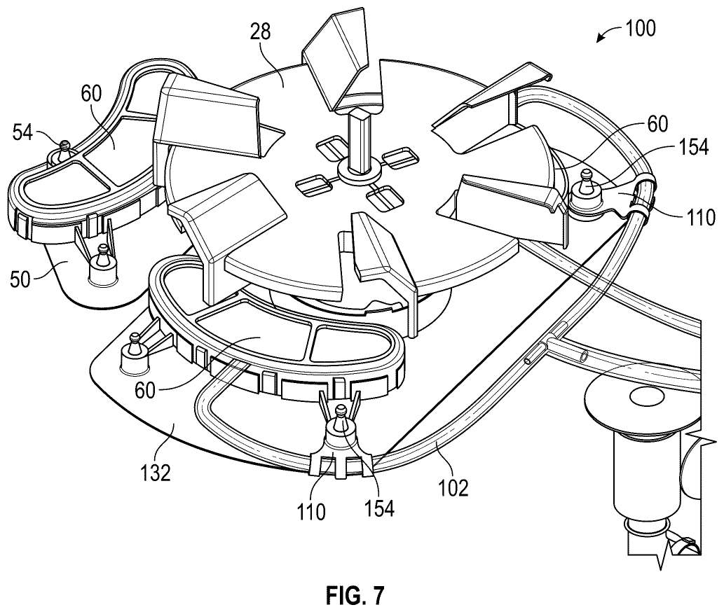

Turning now to FIG. 7, a modular sparging assembly 100 for use with a bioprocessing system (e.g., bioprocessing system 10) according to an exemplary embodiment of the invention is illustrated. As shown therein, the assembly 100 includes a first base plate/impeller base plate 132 having an impeller mounting element/hub for receiving impeller 28 for rotation thereon, and a plurality of mounting elements in the form of mounting posts 154 extending upwardly therefrom. In an embodiment, the impeller base plate 132 is generally rectangular in shape and opposing ends of the base plate 132 on opposite sides of the impeller include an array of mounting posts 154 for receiving respective sparge pods 60 thereon. The impeller base plate 132 is affixed to the bottom of a flexible bioprocessing bag (not shown) in a manner heretofore known in the art. As illustrated, the first base plate 132 thus supports the impeller 28 and two sparge pods 60.

As further shown in FIG. 7, the modular sparging assembly 100 also includes a second base plate/sparger base plate 50 having a plurality of mounting elements in the form of mounting posts 54 extending upwardly therefrom. The sparger base plate 50 is affixed to the bottom of a flexible bioprocessing bag (not shown) in a manner similar to that of the impeller base plate 132. As shown in FIG. 7, the sparger base plate 50 is spaced from, and is mechanically separate from, the first base plate 132, and receives a third sparge pod 60 thereon in the manner hereinbefore described (i.e., on mounting posts 54). Accordingly, this configuration allows the sparger base plate 50, and the sparge pod 60 mounted thereto, to be positioned in the flexible bioprocessing bag 20 at a location that is not dependent on the location of the impeller base plate 132 within the bag 20. That is, independent sparger base plate 50 allows the associated sparge pod 60 to be positioned at a location within the processing volume irrespective of the location of the impeller base plate 132 and the impeller 28. As shown therein, sparge tubing 102 is connected to each sparge pod 60 for supplying sparge gas to each pod 60. While FIG. 7 shows the use of a single sparger base plate 50, additional sparger base plates 50 may be utilized dependent on application and customer needs, which can each be located at any desired position within the flexible bag 20.

Lastly, tubing clips 110 may be utilized to retain the sparge tubing 102 to inhibit the sparge tubing 102 from moving around during processing operations, as discussed in detail below. In an embodiment the tubing clips 110 have a first end that receives the sparge tubing 102, and a second end that can be connected to the mounting posts 54, 154 using a snap-fit connection.

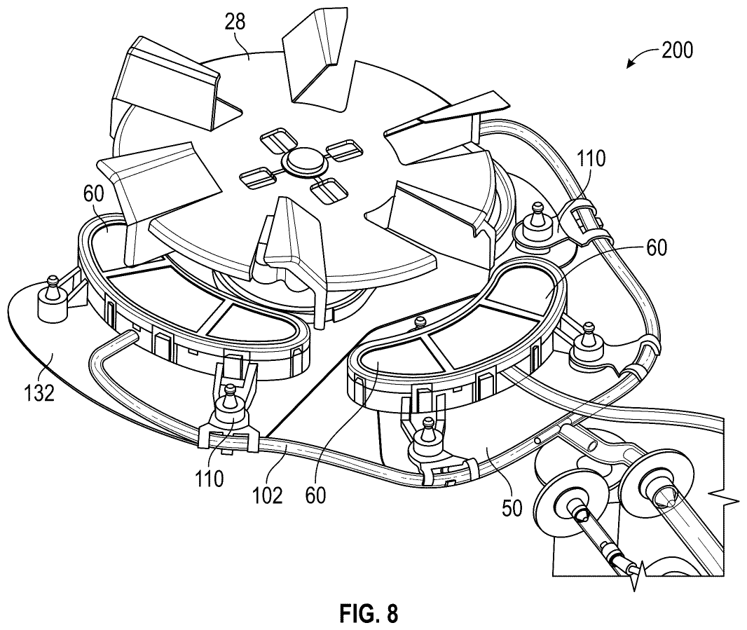

FIG. 8 illustrates a similar modular sparging assembly 200 according to another embodiment of the invention. As shown therein, the assembly 200 includes a pair of sparge pods 60 mounted to the impeller base plate 132 along with impeller 28, and a third sparge pod 60 mounted to independent base plate 50. Rather than being positioned towards the rear of the vessel 12 like the assembly 100, the sparger base plate 50 and associated sparger 60 is located towards the front of the vessel 12 where the sensors and other tubing enters the flexible bag 20.

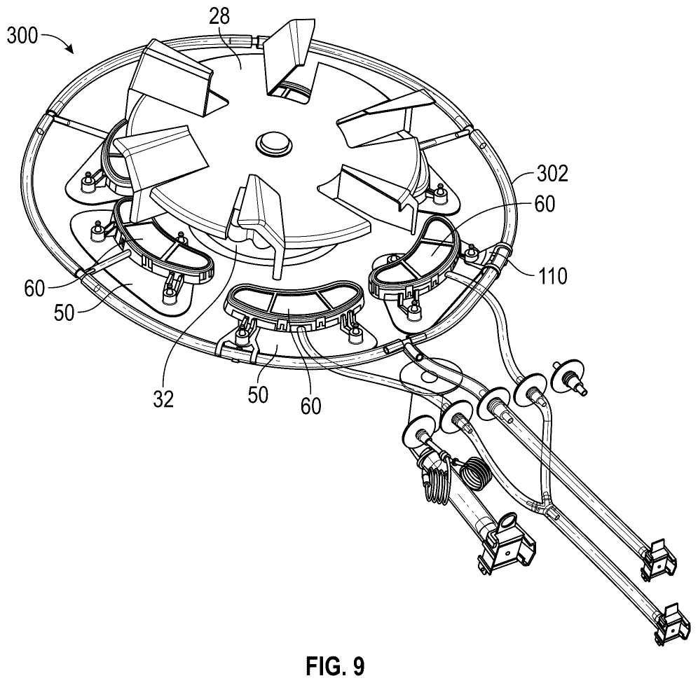

Turning now to FIG. 9, yet another embodiment of a modular sparging assembly 300 is illustrated. As shown therein, the sparging assembly 300 has an impeller base plate 32 having impeller 28 received thereon. As shown, the impeller base plate 32 only supports the impeller 28 and is devoid of any sparging elements. The sparging assembly 300 further includes a plurality of sparger base plates 50 and associated sparge pods 60 arranged in a generally annular configuration around the impeller base plate 32. The sparge pods 60 are supplied with a sparge gas via sparge tubing 302 that forms a ring around the sparge pods 60. The use of the independent sparger base plates 50 allows the number of sparge pods 60, and the distance of the sparge pods 60 from the impeller 28, to be selected according to application, customer requirements, etc. This is in contrast to existing systems whereby the distance of the sparger elements from the impeller is fixed and cannot be varied. As indicated above, tubing clips 110 are utilized to secure the sparge tubing 302 to the sparger base plates 50.

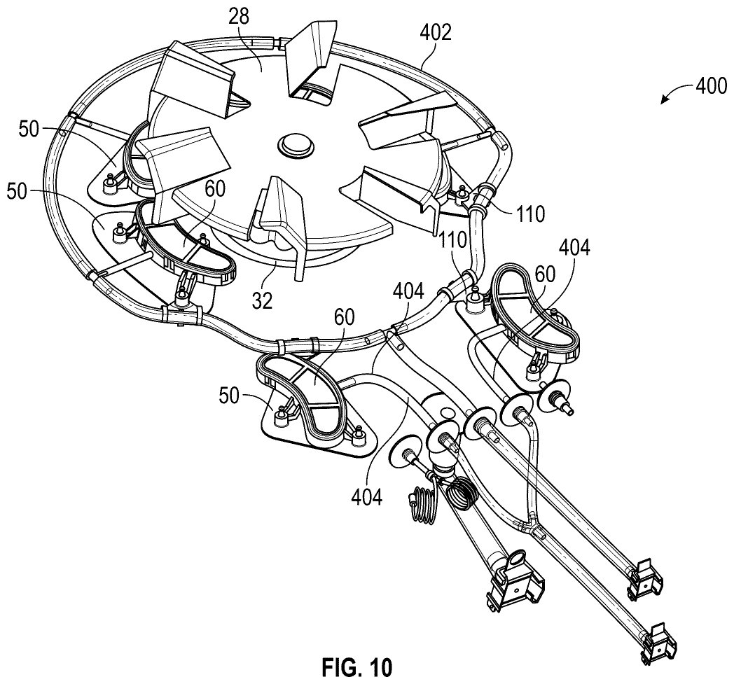

FIG. 10 illustrates another embodiment of a modular sparging assembly 400. As shown therein, a plurality of sparger base plates 50 and associated sparge pods 60 are positioned in a semi-annular configuration around the impeller base plate 32 and impeller 28 and are supplied with gas via sparge tubing 402, while two additional sparger base plates 50 and associated sparge pods 60 are spaced further from the impeller 28 at the front side of the bioprocessing vessel 12 and are supplied with gas via sparge tubing 404. In an embodiment, the gas supplied to sparge pods 60 via sparge tubing 402 may be the same or different than the gas supplied to sparge pods 60 via sparge tubing 404.

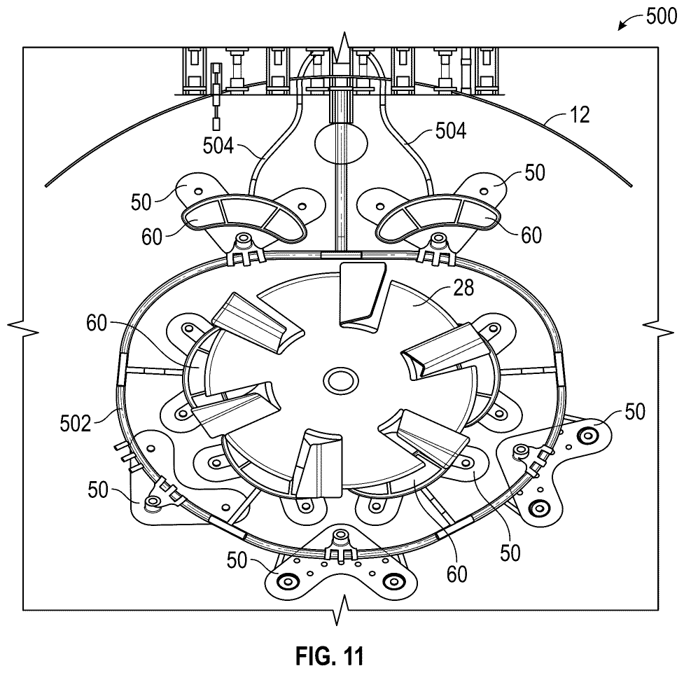

Lastly, FIG. 11 illustrates yet another embodiment of a modular sparging assembly 500. As shown therein, a plurality of sparger base plates 50 and associated sparge pods 60 are positioned in a semi-annular configuration around the impeller base plate 32 and impeller 28, forming an inner ring of sparge pods, and are supplied with gas via sparge tubing 502, while two additional sparger base plates 50 and associated sparge pods 60 are spaced further from the impeller 28 at the front side of the bioprocessing vessel 12 and are supplied with gas via sparge tubing 504. An outer ring of sparger base plates 50 may be provided for the connection of additional sparge pods 60, or for providing additional connection points for sparge tubing 502.

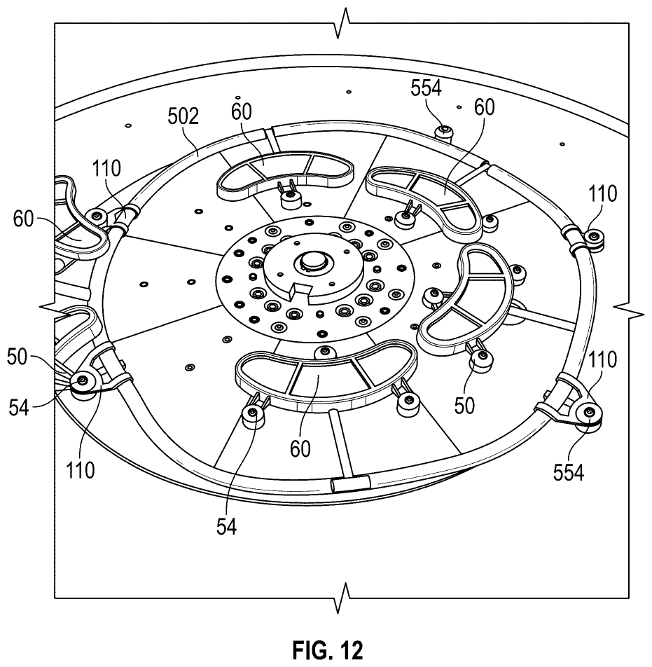

Turning now to FIG. 12, another sparge tubing fixturing arrangement is shown. As illustrated therein, four tubing clips 110 are utilized to maintain the position of the sparge tubing 502, which is arranged in a ring and entirely encircles the impeller. On the left side of the figure, tubing clips 110 are received on the mounting posts 54 of the sparger base plates 50. The other tubing clips 110, however, are engaged with standalone mounting posts 554 that are affixed to the bottom of the flexible bag 20 outboard of the sparge pods 60. As such, the sparge tubing 502 is retained using tubing clips 110 that are engaged with the sparger base plates 50, as well as tubing clips 110 that are engaged with standalone mounting posts 554 (i.e., mounting posts not associated with a sparger base plate). These standalone mounting posts can be affixed to the bag 20 by any means known in the art, such as welding and the like, and in any location to provide reliable and secure fixturing of the sparge tubing 502.

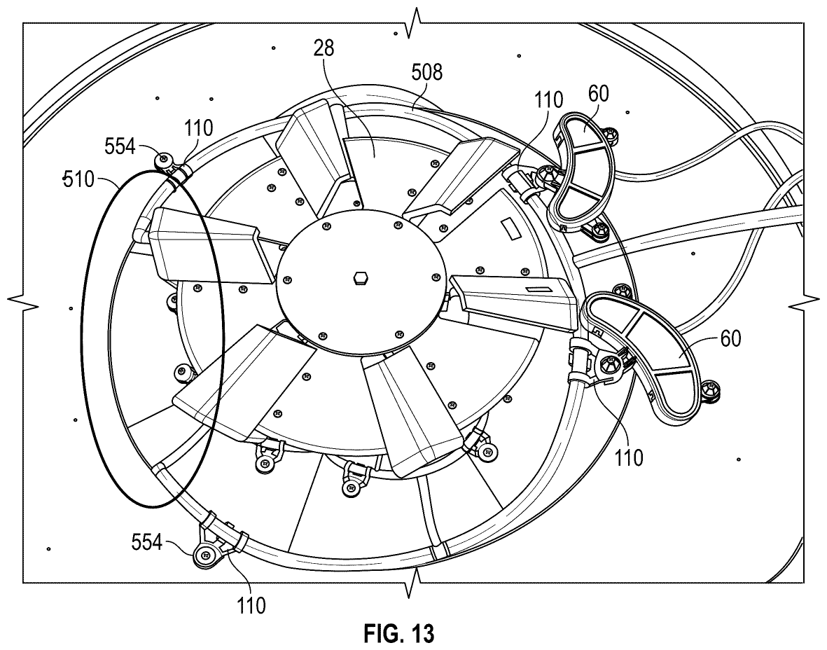

FIG. 13 shows a similar fixturing arrangement, but where the sparge tubing 508 is arranged so as to not extend entirely around the impeller 28. Rather, as shown therein, the sparge tubing 508 extends around opposite sides of the impeller 28, and has an area 510 where sparge tubing is not present.

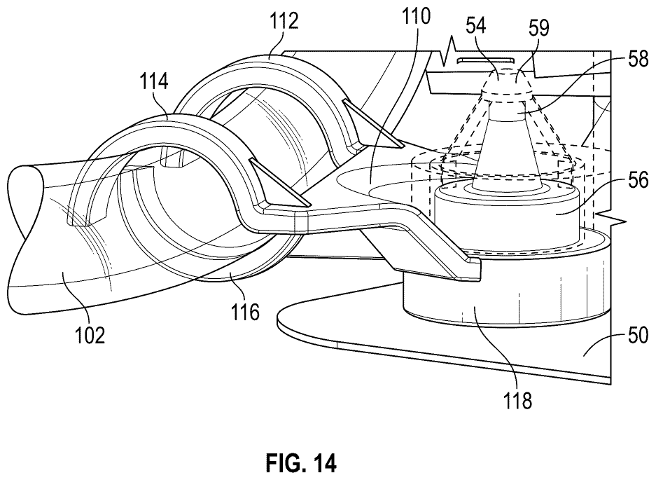

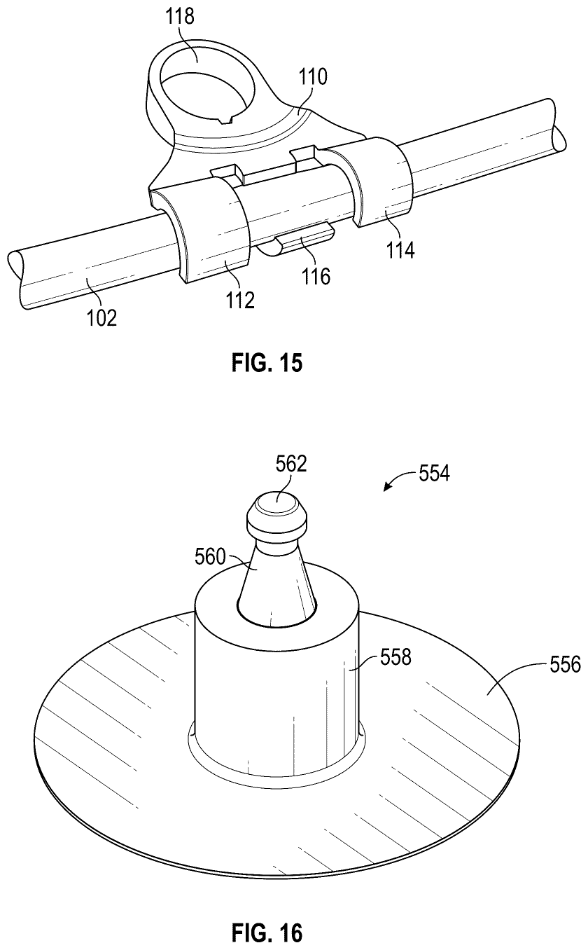

Turning now to FIGS. 14 and 15, the sparge tubing fixturing system described above is better shown. As shown therein, the fixturing system/arrangement includes a mounting post 54 of the sparger base plate 50 that includes a wide, generally cylindrical base 56, and a narrower distal end 58, as described above. In an embodiment, the narrow distal end 58 may include an enlarged head 59 that functions to retain the tubing clip 110 and/or sparge pod 60 thereon once pressed onto the mounting post 54. As best shown in FIG. 15, the tubing clip 110 includes a plurality of resilient arm members 112, 114, 16 that define a generally cylindrical channel therethrough. In an embodiment, the outer arm members 112, 114 have downwardly curved distal ends configured to receive the sparge tubing 102, while the middle arm member 116 has an upwardly curved distal end configured to likewise receive the sparge tubing 102. Other arrangements are also possible. In use, the resilient arm members 112, 114, 116 can be deformed so that the sparge tubing 102 can be captured thereby. As further shown in FIG. 15, an opposite end of the tubing clip 110 includes a generally cylindrical receiving portion 118. As shown in FIG. 14, the receiving portion 118 is received over the mounting post 54, of the sparger base plate 50, and the foot 64 of the sparge pod is snapped onto the mounting post when attaching the sparge pod 60, which affixes the tubing clip 110 to the mounting post 54.

As shown in FIG. 16, and as indicated above, instead of, or in addition to the mounting posts 54 of the sparger base plates 50, standalone mounting posts 554 may be used at various locations within the bioprocessing bag 20 to receive and secure the tubing clips 110 and associated sparge tubing 102. As shown therein, in an embodiment, the standalone mounting posts 554 may include a flat base 556 that is welded or otherwise fixed to the flexible bag 20, a post having an enlarged base 558, and a narrower distal end 560. In an embodiment, the narrow distal end 560 may include an enlarged head 562 that functions to retain the tubing clip 110 thereon once pressed onto the mounting post 554. As disclosed above, the flat base 556 can be affixed to the bottom of the flexible bag by any means known in the art, such as welding and the like.

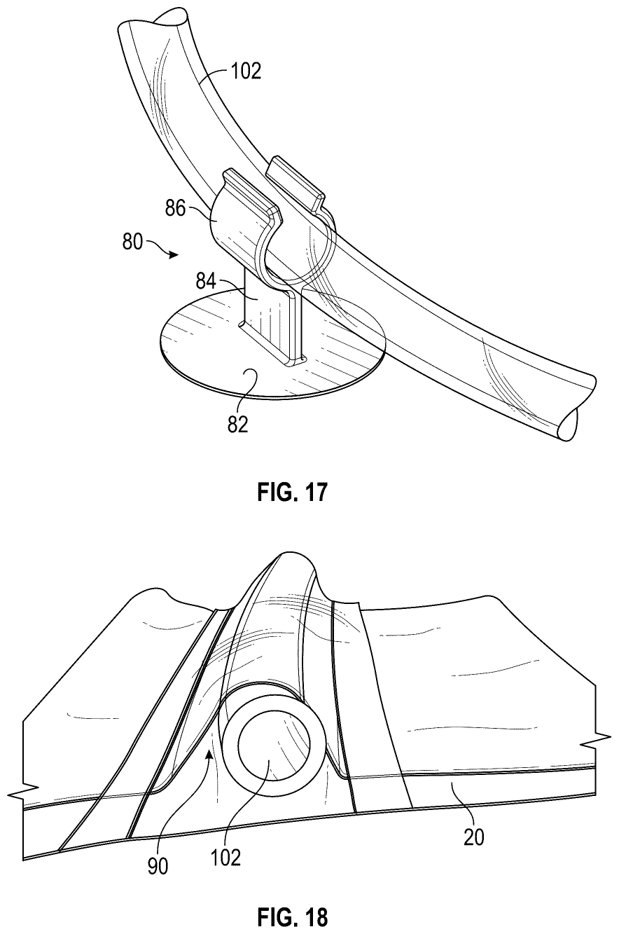

FIG. 17 illustrates a tubing retainer 80 for retaining the sparge tubing 102 and inhibit it from moving around during operation of the impeller 28, according to another embodiment of the invention. Tubing retainer 80 can be utilized instead of, or in addition to, mounting posts 54, 554. As shown in FIG. 17, the tubing retainer includes a flat base 82, an upright 84 extending upwardly from the base, and a generally U-shaped, resilient tubing clip 86 having an open upper end configured to receive the sparge tubing 102 therein. As with the standalone mounting posts 554, a plurality of tubing retainers 80 can be affixed to the bottom of the flexible bag 20 utilizing any means known in the art, and in any desired location so as to retain and inhibit movement of the sparge tubing 102 during operation of the bioprocessing system 10.

Turning finally to FIG. 18, in combination with, or alternative to, any of the sparge tubing fixturing arrangements disclosed above, in an embodiment, the flexible bag 20 may be formed with internal channels 90 through which the sparge tubing 102 can be positioned. These channels 90 may be formed, for example, by welding or adhering a ply of bag material to the bottom of the flexible bag 20. As shown in FIG. 18, the internal channels 90 retain the sparge tubing 102 and prevent it from moving within the flexible bag 20 during bioprocessing operations. In contrast to the embodiments disclosed above, the internal channels 90 fully isolate the sparge tubing 102 from processing environment within the flexible bag 20. It is further envisioned that in certain embodiments, that the tubing 102 may be omitted, and the sparge gas may flow directly through the internal channels 90.

As indicated above, the use of a separate impeller base plate 32 and multiple sparger base plates 50 allows for the positioning of sparger elements at any desired location within the processing volume, irrespective of where the impeller base plate 32 and impeller 28 are located. For example, this configuration allows for the positioning of multiple sparger elements beneath the impeller 28, as well as other spargers offset from the impeller 28. This configuration also allows for the placement of any number of sparger elements (e.g., between 1 and 6 or more) within the processing volume to meet customer applications and needs. The sparge tubing fixturing mechanisms disclosed herein also provide secure and reliable retention of the sparge tubing and vibration damping, which minimizes the possibility that the sparge tubing can oscillate during bioprocessing operations and compromise the integrity of internal components. Still further, this configuration allows for user customization without the need for custom manufacturing (i.e., the mold used for the base plates does not change even though the number and location of sparger elements is customizable).

In an embodiment, a sparging assembly is provided. The sparging assembly includes a first base plate having at least one mounting element for receiving a first sparger device, and a second base plate having at least one mounting element for receiving a second sparger device. The first base plate and the second base plate are mechanically separate from one another. In an embodiment, the first base plate includes an impeller mounting element for connecting an impeller to the first base plate. In an embodiment, the first base plate and the second base plate are configured for connection to an interior of a flexible bioprocessing container. In an embodiment, the at least one mounting element of the first base plate and the at least one mounting element of the second base plate are a plurality of posts extending upward from the first base plate and the second base plate, respectively. In an embodiment, the plurality of posts is three posts. In an embodiment, the sparging assembly further includes at least one tubing retainer configured to receive and retain sparge tubing that supplies a gas to at least one of the first sparger device and the second sparger device. In an embodiment, the at least one tubing retainer is mounted to the first base plate or the second base plate. In an embodiment, the at least one tubing retainer is configured for connection to the flexible bioprocessing vessel. In an embodiment, the tubing retainer is a clip.

According to another embodiment, a bioprocessing system is provided. The bioprocessing system includes a bioprocessing container, a plurality of base plates mounted within an interior of the bioprocessing container, at least one of the plurality of base plates being mechanically separate from another of the plurality of base plates, and a plurality of sparger devices mounted to the plurality of base plates. In an embodiment, the bioprocessing container is a flexible bioprocessing bag. In an embodiment, the plurality of base plates include a first base plate and a second base plate, and the plurality of sparger devices include a first sparger device mounted to the first base plate and a second sparger device mounted to the second base plate. The bioprocessing system further includes an impeller mounted to the first base plate. In an embodiment, the bioprocessing system further includes an impeller base plate, and an impeller mounted to the impeller base plate, wherein the plurality of base plates and the plurality of spargers are arranged around the impeller base plate. In an embodiment, the plurality of based plates are 6 base plates, each of the base plates being mechanically separate from one another, and the plurality of sparger devices are 6 sparger devices. In an embodiment, the plurality of base plates is between 3 and 6 base plates, each having an associated sparger device of the plurality of sparger devices. In an embodiment, the bioprocessing system further includes at least one tubing retainer configured to receive and retain sparge tubing that supplies a gas to the plurality of sparger devices. The at least one tubing retainer may be mounted to one of the plurality of base plates. In an embodiment, the bioprocessing container is a flexible bioprocessing bag, and the at least one tubing retainer is mounted to an interior of the flexible bioprocessing vessel. In an embodiment, the flexible bioprocessing bag includes an integrated channel through which sparge gas is provided to the plurality of sparger devices. In an embodiment the bioprocessing system may further include a rigid support vessel, wherein the flexible bioprocessing bag is received within the rigid support vessel.

According to yet another embodiment of the invention, a method of configuring a bioprocessing system is provided, and includes the steps of affixing an impeller base plate to an interior of a flexible bioprocessing bag, mounting an impeller to the impeller base plate, affixing a first sparger base plate to the interior of the flexible bioprocessing bag adjacent to the impeller base plate, and mounting a first sparger device to the first sparger base plate. The first sparger base plate and the impeller base plate are spaced from one another. In an embodiment, the method may also include the steps of affixing a second sparger base plate to the interior of the flexible bioprocessing bag adjacent to the impeller base plate, and mounting a second sparger device to the second sparger base plate, wherein the second sparger base plate is spaced from the impeller base plate and the first sparger base plate.

As used herein, an element or step recited in the singular and proceeded with the word “a” or “an” should be understood as not excluding plural of said elements or steps, unless such exclusion is explicitly stated. Furthermore, references to “one embodiment” of the invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A sparging assembly, comprising:
a first base plate having at least one mounting element for receiving a first sparger device; and
a second base plate having at least one mounting element for receiving a second sparger device;
wherein the first base plate and the second base plate are mechanically separate from one another.

2. The sparging assembly of claim 1, wherein:
the first base plate includes an impeller mounting element for connecting an impeller to the first base plate.

3. The sparging assembly of claim 1, wherein:
the first base plate and the second base plate are configured for connection to an interior of a flexible bioprocessing container.

4. The sparging assembly of claim 1, wherein:
the at least one mounting element of the first base plate and the at least one mounting element of the second base plate are a plurality of posts extending upward from the first base plate and the second base plate, respectively.

5. The sparging assembly of claim 4, wherein:
the plurality of posts is three posts.

6. The sparging assembly of claim 2, further comprising:
at least one tubing retainer configured to receive and retain sparge tubing that supplies a gas to at least one of the first sparger device and the second sparger device.

7. The sparging assembly of claim 6, wherein:
the at least one tubing retainer is mounted to the first base plate or the second base plate.

8. The sparging assembly of claim 6, wherein:
the at least one tubing retainer is configured for connection to the flexible bioprocessing vessel.

9. The sparging assembly of claim 6, wherein:
the tubing retainer is a clip.

10. A bioprocessing system, comprising:
a bioprocessing container;
a plurality of base plates mounted within an interior of the bioprocessing container, at least one of the plurality of base plates being mechanically separate from another of the plurality of base plates; and
a plurality of sparger devices mounted to the plurality of base plates.

11. The bioprocessing system of claim 10, wherein:
the bioprocessing container is a flexible bioprocessing bag.

12. The bioprocessing system of claim 10, wherein:
the plurality of base plates include a first base plate and a second base plate; and
the plurality of sparger devices include a first sparger device mounted to the first base plate and a second sparger device mounted to the second base plate;
wherein the bioprocessing system further includes an impeller mounted to the first base plate.

13. The bioprocessing system of claim 10, further comprising:
an impeller base plate; and
an impeller mounted to the impeller base plate;
wherein the plurality of base plates and the plurality of spargers are arranged around the impeller base plate.

14. The bioprocessing system of claim 13, wherein:
the plurality of based plates are 6 base plates, each of the base plates being mechanically separate from one another; and
the plurality of sparger devices are 6 sparger devices.

15. The bioprocessing system of claim 10, wherein:
the plurality of base plates is between 3 and 6 base plates, each having an associated sparger device of the plurality of sparger devices.

16. The bioprocessing system of claim 10, further comprising:
at least one tubing retainer configured to receive and retain sparge tubing that supplies a gas to the plurality of sparger devices.

17. The bioprocessing system of claim 16, wherein:
the at least one tubing retainer is mounted to one of the plurality of base plates.

18. The bioprocessing system of claim 16, wherein:
the bioprocessing container is a flexible bioprocessing bag; and
wherein the at least one tubing retainer is mounted to an interior of the flexible bioprocessing vessel.

19. The bioprocessing system of claim 11, wherein:
the flexible bioprocessing bag includes an integrated channel through which sparge gas is provided to the plurality of sparger devices.

20. The bioprocessing system of claim 11, further comprising:
a rigid support vessel;
wherein the flexible bioprocessing bag is received within the rigid support vessel.

21. A method of configuring a bioprocessing system, comprising the steps of:
affixing an impeller base plate to an interior of a flexible bioprocessing bag;
mounting an impeller to the impeller base plate;
affixing a first sparger base plate to the interior of the flexible bioprocessing bag adjacent to the impeller base plate; and
mounting a first sparger device to the first sparger base plate;
wherein the first sparger base plate and the impeller base plate are spaced from one another.

22. The method according to claim 21, further comprising the step of:
affixing a second sparger base plate to the interior of the flexible bioprocessing bag adjacent to the impeller base plate; and
mounting a second sparger device to the second sparger base plate;
wherein the second sparger base plate is spaced from the impeller base plate and the first sparger base plate.
